(19) United States
(12) Patent Application Publication
(10) Pub. No.: US 2025/0011266 A1
(43) Pub. Date: Jan. 9, 2025

(54) METHODS OF MAKING CANNABIDIVARIN ISOLATE

(71) Applicant: BioPyure, LLC, Arvada, CO (US)

(72) Inventors: George Clampitt, Arvada, CO (US); Harrison Stout, Arvada, CO (US)

(21) Appl. No.: 18/890,351

(22) Filed: Sep. 19, 2024

Related U.S. Application Data
(60) Provisional application No. 63/684,173, filed on Aug. 16, 2024.

(51) Int. Cl. C07C 37/84

(52) U.S. Cl. CPC C07C 37/84; C07B 2200/13; C07C 2601/16

(57) ABSTRACT

A method of preparing a cannabidivarin (CBDv) isolate generally includes a step of dissolving a distillate comprising (cannabidivarin), such as a full spectrum distillate obtained from hemp extract, in alkane solvent to form a first solution, cooling the first solution to within a first temperature range to thereby form first crystals comprising CBDv, dissolving the first crystals in alkane solvent to form a second solution, cooling the second solution to within a second temperature range to thereby form second crystals having a higher CBDv content than the first crystals, dissolving the second crystals in alkane solvent to form a third solution, and cooling the third solution to within a third temperature range to thereby form third crystals having a higher CBDv content than the second crystals. The method can be carried out to prepare crystals having a CBDv content greater than 97 wt %, such as greater than 99 wt % or greater than 99.9 wt %.

### BACKGROUND

Cannabidivarin (CBDv) is a cannabinoid found in hemp plants. Recent studies have suggested that CBDv may demonstrate anti-epileptic and/or anti-convulsive action when administered to humans. As such, recent attention has focused on isolating CBDv to provide compositions having high CBDv concentrations. However, to date, isolation techniques for isolating CBDv from, for example, full spectrum distillates derived from hemp plants, are costly, complex, and/or inefficient. Accordingly, a need exists for improved methods of making CBDv isolate.

### SUMMARY

In some embodiments, a method of preparing a cannabidivarin (CBDv) isolate includes a first crystallization step, a second crystallization step, and a third crystallization step. The first crystallization step may include dissolving a distillate comprising CBDv, such as a full spectrum distillate obtained from hemp extract, in alkane solvent to form a first solution, and cooling the first solution to form first crystals having a CBDv content. The second crystallization step may include dissolving the first crystals in alkane solvent to form a second solution, and cooling the second solution to form second crystals having a CBDv content greater than the CBDv content of the first crystals. The third crystallization step may include dissolving the second crystals in alkane solution to form a third solution, and cooling the third solution to form third crystals having a CBDv content greater than the CBDv content of the second crystals. The second and third crystallization steps may be repeated one or more times. The ratio of distillate or crystals to alkane used in the dissolving steps may vary and/or the temperature to which the solutions are cooled may vary from crystallization step to crystallization step. In some embodiments, the third crystallization step is repeated until the CBDv content of the crystals formed in the third crystallization step is greater than 97 wt %, such as greater than 99 wt % or greater than 99.9 wt %.

In some embodiments, a method of preparing a CBDv isolate includes a purification step and a crystallization step. The purification step may include performing one or more purification processes on a distillate comprising CBDv (such as a full spectrum distillate obtained from hemp extract). The one or more purification processes are carried until the distillate has a CBDv content greater than a threshold amount, such as higher than 80 wt % CBDv. The crystallization step generally includes dissolving the purified distillate in alkane solvent and then cooling the solution to form crystals with high CBDv content. In some embodiments, the crystallization step is repeated one or more times in order to form crystals with a desired CBDv content, such as greater than 97 wt %.

### BRIEF DESCRIPTION OF THE DRAWINGS

Non-limiting and non-exhaustive embodiments of the disclosed technology, including the preferred embodiment, are described with reference to the following figures, wherein like reference numerals refer to like parts throughout the various views unless otherwise specified.

FIG. 1 is a flow chart illustrating a method of making cannabidivarin (CBDv) isolate in accordance with various embodiments described herein.

FIG. 2 is a flow chart illustrating a method of making cannabidivarin isolate in accordance with various embodiments described herein.

### DETAILED DESCRIPTION

Embodiments are described more fully below with reference to the accompanying Figures, which form a part hereof and show, by way of illustration, specific exemplary embodiments. These embodiments are disclosed in sufficient detail to enable those skilled in the art to practice the invention. However, embodiments may be implemented in many different forms and should not be construed as being limited to the embodiments set forth herein. The following detailed description is, therefore, not to be taken in a limiting sense.

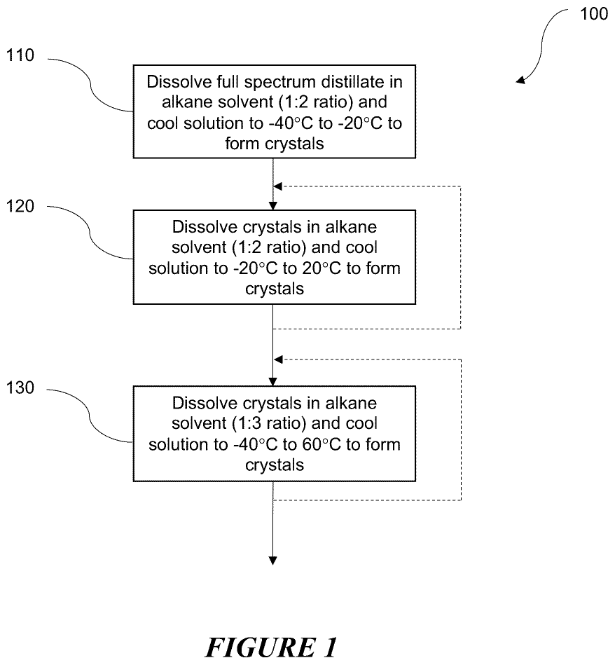

FIG. 1 illustrates a method of making a cannabidivarin (CBDv) isolate in accordance with various embodiments described herein. The method 100 generally includes a step 110 wherein a full spectrum extract or distillate is dissolved in a solvent and then cooled to form first crystals; a step 120 where the first crystals are dissolved in a solvent and then cooled to form second crystals; a step 130 where the second crystals are dissolved in a solvent than cooled to form third crystals. In some embodiments, step 120 is repeated more than once, such as two times, before proceeding to step 130. In some embodiments, step 130 is repeated more than once, such as repeating step 130 as many times as necessary until a CBDv isolate with a desired level of purity is obtained.

In step 110, a distillate is provided and subjected to initial processing that results in the creation of CBD/CBDv crystals and the separation/removal of other components of the full spectrum distillate. The specific composition of the distillate used in step 110 is generally not limited, though the distillate will generally include any combination of various cannabinoids (including cannabidiol (CBD) and CBDv), chlorophyl, flavonoids, terpenes, fats, waxes, sugars, and/or salts. The distillate used in step 110 may be a composition obtained from an extract of a hemp plant, such as when an extract is subjected to distillation to target and increase the concentration of specific components of the extract and thereby form a distillate. The hemp extract from which the distillate is derived is generally obtained from processing a hemp plant to separate components of the hemp plant from the plant biomass, typically in the form a solution. Any known extraction and/or separation techniques can be used to process the hemp plant and form an extract, including, but not limited to, solvent extraction and extraction using supercritical CO₂. Any known distillation techniques can be used to obtain a distillate from the hemp extract. In some embodiments, the distillate used in step 110 is a full spectrum distillate.

As part of step 110, the distillate is mixed with a solvent to form a solution wherein the distillate (including all components thereof) is fully dissolved in the solvent. In some embodiments, the solvent used in step 110 is an alkane. Any manner of combining or mixing the distillate with the solvent can be used provided that the distillate is fully incorporated into the solution.

In some embodiments the ratio of distillate to alkane solvent used in step 110 is any suitable ratio that allows for the components of the distillate to be dissolved in the alkane solvent. In some embodiments, the ratio is about 1:2, wherein the amount of distillate is measured in kilograms and the amount of alkane solvent is measured in liters. Thus, in one example, 5 kg distillate is mixed with 10 L of alkane solvent in step 110. Once combined in the desired ratio, additional steps, such as mechanical mixing or agitation, may be carried out to ensure the components of the distillate become fully dissolved in the alkane solvent.

Once the solution of fully dissolved distillate in alkane solvent is prepared, the solution is subjected to cooling. In some embodiments, the solution is cooled to a temperature between −40° C. and 60° C., such as between −40° C. and −20° C. Any manner of cooling the solution can be used, and any rate of cooling can be used.

Once the solution has been cooled to within the desired temperature range, crystals will form in the solution. In some embodiments, the cooled solution is allowed to rest for a period of time following cooling to allow for a maximum amount of crystal formation.

Once the rest period has passed, the formed crystals may be separated from the solution. To separate the formed crystals from the solution, filtration and washing are generally used. The filtration step generally uses any suitable filter and/or filtration techniques that allows for the solution to pass therethrough while preventing crystals from passing therethrough. Once the liquid solution has been substantially separated from the crystals via filtration, the crystals may be washed in order to rinse from the crystals any residual solution. In some embodiments, the same alkane solvent used to dissolve the full spectrum distillate or extract is used to carry out the washing step. The alkane used for the wash step is preferably a cold alkane.

The crystals formed in step 110 generally comprise CBD and CBDv, with only trace amounts of other components from the full spectrum distillate or extract. In some embodiments, the composition of the crystals is from about 50 to 80 wt % CBDv and from about 20 to 50 wt % CBD.

In step 120, the crystals obtained from step 110 are mixed with alkane solvent to fully dissolve the crystals in the alkane solvent and form a solution, followed by cooling the solution, and allowing for crystal formation following cooling. In this manner, step 120 is similar to step 110, although some of the processing conditions are adjusted for step 120 as discussed in further detail below.

Step 120 generally begins by mixing the crystals formed in step 110 with an alkane solvent to fully dissolve the crystals in the alkane solvent. In some embodiments, the alkane solvent used in step 120 is the same alkane solvent used in step 110. Furthermore, the ratio of crystals to alkane solvent (on a kg to L basis) may be the same as used in step 110. Similar to step 110, mixing crystals and alkane solvent in step 120 is carried out in a manner that ensures the crystals fully dissolve in the alkane solvent, such as by promoting dissolution via mixing and/or agitation.

Following full dissolution, step 120 proceeds by cooling the solution. In some embodiments, the cooling portion of step 120 differs from the cooling portion of step 110 in that cooling in step 120 is carried out to cool the solution to a temperature in the range of from −40° C. and 60° C., such as between −20° C. to 20° C. As with the cooling portion of step 110, the cooling portion of step 120 may be carried out using any suitable cooling techniques and/or equipment, and may be carried out for any period of time and at any suitable cooling rate.

Following cooling to a temperature in the range of from, for example, −20° C. to 20° C., the solution is allowed to rest for a period of time that allows for the full extent of crystallization formation. Once complete, or at least a desired amount of crystallization occurs, the formed crystals are subjected to filtration and washing similar or identical to the filtration and washing steps described previously with respect to step 110. As such, filtration allows the formed crystals to be separated from the solution, and washing with cool alkane allows for any residual solution to be washed from the crystals.

In some embodiments, the crystals formed from step 120 are about 80 wt. % CBDv, with the remainder being CBD (and potentially trace amounts of other components).

In some embodiments, step 120 is repeated at least one time, though step 120 need not be repeated, or may be repeated more than one time. When step 120 is repeated one or more times, the outcome is the formation of crystals with an even higher content of CBDv.

In step 130, the crystals formed in step 120 are again dissolved in an alkane solvent, followed by cooling the solution and allowing for crystal formation. In this manner, step 130 is similar to steps 110 and 120, although some of the processing conditions are adjusted for step 130 as discussed in further detail below.

Step 130 generally begins by mixing the crystals formed in step 120 with an alkane solvent to fully dissolve the crystals in the alkane solvent. In some embodiments, the alkane solvent used in step 130 is the same alkane solvent used in steps 110 and 120. However, in step 130, the ratio of crystals to alkane solvent (on a kg to L basis) may be different from the ratio used in step 110 and/or step 120. In some embodiments, more alkane solvent in L per kg crystal is used in step 130 than in steps 110 and 120. In one non-limiting example, the ratio used in step 130 is about 1:3. Regardless of the different ratio of crystals to solvent used in step 130, mixing crystals and alkane solvent in step 130 is still carried out in a manner that ensures the crystals fully dissolve in the alkane solvent, such as by promoting dissolution via mixing and/or agitation. In some embodiments, full dissolution of the crystals into the solvent is promoted in step 130 by raising the temperature of the mixture of crystals and solvent during and/or after mixing.

Following full dissolution, step 130 proceeds by cooling the solution. In some embodiments, the cooling portion of step 130 is similar or identical to the cooling portion of step 120, i.e., cooling is carried out to cool the solution to a temperature in the range of from −40° C. and 60° C., such as between −20° C. to 20° C. The cooling portion of step 130 may be carried out using any suitable cooling techniques and/or equipment, and may be carried out for any period of time and at any suitable cooling rate.

Following cooling to a temperature in the range of from, for example, −20° C. to 20° C., the solution is allowed to rest for a period of time that allows for the full extent of crystallization formation. Once complete, or at least a desired amount of crystallization occurs, the formed crystals are subjected to filtration and washing similar or identical to the filtration and washing steps described previously with respect to step 120. As such, filtration allows the formed crystals to be separated from the solution, and washing with cool alkane allows for any residual solution to be washed from the crystals.

Following the filtration and washing portion of step 130, the resulting crystals may have a CBDv content greater than the CBDv content of crystals formed in step 120. In some embodiments, in order to further increase the CBDv content in the crystals, step 130 is repeated one or more times. With each successive iteration of step 130, the content of CBDv content in the produced crystals increases. In some embodiments, step 130 is repeated until the crystals produced have a CBDv content of greater than 97 wt %, such as greater than 98 wt %, greater than 99 wt %, greater than 99.5 wt %, greater than 99.7 wt %, or greater than 99.9 wt %. In some embodiments, step 130 is repeated until the CBD content of the crystals is at least less than 0.3 wt %. In some embodiments, step 130 is repeated until the CBD content of the crystals is non-detectable.

In an optional post processing step (i.e., after performing step 130 enough times to obtain the desired level of CBDv content in the crystals), the crystals may be dried under heat and/or vacuum to remove any residual solvent. It is important to remove residual solvent to ensure the CBDv crystal product is safe for consumer consumption.

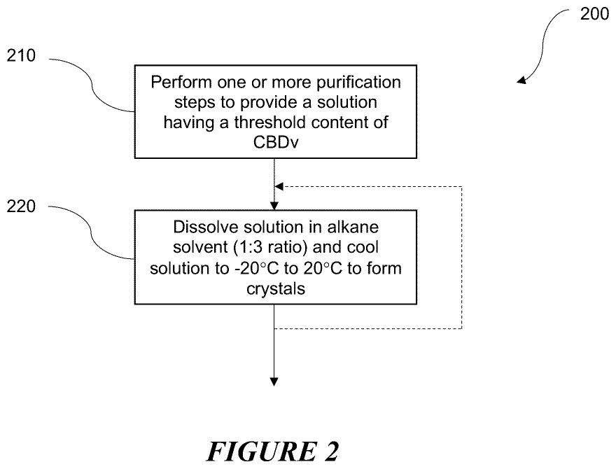

With reference to FIG. 2, a method 200 for preparing an CBDv isolate is illustrated, with method 200 differing from method 100 described previously primarily with respect to the preliminary purification steps. Method 200 generally includes a step 210 wherein a distillate is subjected to any suitable purification process to thereby increase the concentration of CBDv in the distillate, and a step 220 wherein the concentrated CBDv composition is dissolved in a solvent than cooled to form crystals. In some embodiments, step 220 is repeated more than once, such as repeating step 220 as many times as necessary until a CBDv isolate with a desired level of purity is obtained.

With respect to step 210, a distillate similar or identical to the distillate described previously with respect to step 110, is provided. The specific composition of the distillate used in step 210 is generally not limited, though the distillate will generally include any combination of various cannabinoids (including cannabidiol (CBD) and CBDv), chlorophyl, flavonoids, terpenes, fats, waxes, sugars, and/or salts. In some embodiments, the distillate used in step 210 is a full spectrum distillate.

In step 210, the distillate is subjected to one or more purification processes that results in producing a solution with an increased concentration of CBDv relative to the concentration of CDBv in the distillate. Any suitable purification processes can be used as part of step 210. In some embodiments, chromatography is used in order to produce a solution having an increased concentration of CBDv. Mechanical purification can also be used in step 210. In some embodiments, the purification used in step 210 excludes the type of purification processing described previously with respect to steps 110, 120, and 130 of method 100. That is to say, the purification of step 210 does not include purification wherein the distillate is dissolved in a solvent and cooled to form CBDv crystals.

Ultimately, the primary aim of step 210 is to use one or more purification processes to prepare a solution having an increased concentration of CBDv relative to the concentration of CBDv in the original distillate. As such, the specific purification process or processes used is less important than the result obtained. In some embodiments, step 210 is carried out to provide a solution having a CBDv content that is higher than a threshold amount. In some embodiments, the threshold amount of CBDv in the solution resulting from step 210 is greater than 80 wt %, such as greater 85 wt %, greater than 90 wt %, or greater than 95%.

Once step 210 has been carried out to provide a solution with at least the threshold content of CBDv (e.g., greater than 80 wt % CBDv), step 220 is carried in order to provide the desired CBDv isolate having a high content of CBDv. Step 220 is generally similar or identical to step 130 described previously. As such, step 220 generally begins by mixing the solution formed in step 210 with an alkane solvent to fully dissolve the solution (and the components thereof) in the alkane solvent. The ratio of solution to alkane solvent (on a kg solution to L alkane basis) used in step 220 is about 1:3. The solution and alkane solvent are combined in a manner that ensures the solution (and components thereof) fully dissolve in the alkane solvent, such as by promoting dissolution via mixing and/or agitation. In some embodiments, full dissolution of the solution into the solvent is promoted in step 220 by raising the temperature of the mixture of solution and solvent during and/or after mixing.

Following full dissolution, step 220 proceeds by cooling the solution. In some embodiments, cooling is carried out to cool the solution to a temperature in the range of from −40° C. and 60° C., such as between −20° C. to 20° C. The cooling portion of step 220 may be carried out using any suitable cooling techniques and/or equipment, and may be carried out for any period of time and at any suitable cooling rate.

Following cooling to a temperature in the range of from, for example, −20° C. to 20° C., the solution is allowed to rest for a period of time that allows for the full extent of crystallization formation. Once complete, or at least a desired amount of crystallization occurs, the formed crystals are subjected to filtration and washing similar or identical to the filtration and washing steps described previously with respect to steps 110, 120 and 130. As such, filtration allows the formed crystals to be separated from the solution, and washing with cool alkane allows for any residual solution to be washed from the crystals.

Following the filtration and washing portion of step 220, the resulting crystals may have a CBDv content greater than the CBDv content of solution formed in step 210. In some embodiments, in order to further increase the CBDv content in the crystals, step 220 is repeated one or more times. With each successive iteration of step 220, the content of CBDv in the produced crystals increases. In some embodiments, step 220 is repeated until the crystals produced have a CBDv content of greater than 97 wt %, such as greater than 98 wt %, greater than 99 wt %, greater than 99.5 wt %, greater than 99.7 wt %, or greater than 99.9 wt %. In some embodiments, step 220 is repeated until the CBD content of the crystals is at least less than 0.3 wt %. In some embodiments, step 220 is repeated until the CBD content of the crystals is non-detectable.

In an optional post processing step (i.e., after performing step 220 enough times to obtain the desired level of CBDv content in the crystals), the crystals may be dried under heat and/or vacuum to remove any residual solvent. It is important to remove residual solvent to ensure the CBDv crystal product is safe for consumer consumption.

From the foregoing, it will be appreciated that specific embodiments of the invention have been described herein for purposes of illustration, but that various modifications may be made without deviating from the scope of the invention. Accordingly, the invention is not limited except as by the appended claims.

Although the technology has been described in language that is specific to certain structures and materials, it is to be understood that the invention defined in the appended claims is not necessarily limited to the specific structures and materials described. Rather, the specific aspects are described as forms of implementing the claimed invention. Because many embodiments of the invention can be practiced without departing from the spirit and scope of the invention, the invention resides in the claims hereinafter appended.

Unless otherwise indicated, all number or expressions, such as those expressing dimensions, physical characteristics, etc., used in the specification (other than the claims) are understood as modified in all instances by the term “approximately”. At the very least, and not as an attempt to limit the application of the doctrine of equivalents to the claims, each numerical parameter recited in the specification or claims which is modified by the term “approximately” should at least be construed in light of the number of recited significant digits and by applying rounding techniques. Moreover, all ranges disclosed herein are to be understood to encompass and provide support for claims that recite any and all sub-ranges or any and all individual values subsumed therein. For example, a stated range of 1 to 10 should be considered to include and provide support for claims that recite any and all sub-ranges or individual values that are between and/or inclusive of the minimum value of 1 and the maximum value of 10; that is, all sub-ranges beginning with a minimum value of 1 or more and ending with a maximum value of 10 or less (e.g., 5.5 to 10, 2.34 to 3.56, and so forth) or any values from 1 to 10 (e.g., 3, 5.8, 9.9994, and so forth).

I/We claim:

1. A method of preparing a cannabidivarin isolate, comprising:
a first crystallization step, comprising:
dissolving a distillate comprising cannabidivarin in alkane solvent to thereby form a first solution; and
cooling the first solution to within a first temperature range, the first temperature range being from about −40° C. to about 60° C. to thereby form first crystals comprising cannabidivarin;
a second crystallization step, comprising:
dissolving the first crystals in alkane solvent to thereby form a second solution; and
cooling the second solution to within a second temperature range, the second temperature range being from about −40° C. to about 60° C. to thereby form second crystals comprising cannabidivarin, the second crystals having a cannabidivarin content greater than the cannabidivarin content of the first crystals; and
a third crystallization step, comprising:
dissolving the second crystals in alkane solvent to thereby form a third solution; and
cooling the third solution to within a third temperature range, the third temperature range being from about −40° C. to about 60° C. to thereby form third crystals comprising cannabidivarin, the third crystals having a cannabidivarin content greater than the cannabidivarin content of the second crystals.

2. The method of claim 1, wherein the distillate is a full spectrum distillate obtained from hemp extract.

3. The method of claim 1, wherein the first temperature range is from about −40° C. to about −20° C.

4. The method of claim 1, wherein the second temperature range is from about −20° C. to 20° C.

5. The method of claim 1, wherein the third temperature range is from about −20° C. to 20° C.

6. The method of claim 1, wherein:
the first crystals are filtered and washed prior to the second crystallization step;
the second crystals are fileted and washed prior to the third crystallization step; and
the third crystals are filtered and washed.

7. The method of claim 1, further comprising:
repeating the second crystallization step one or more times prior to the third crystallization step, wherein the crystals formed at the conclusion of each second crystallization step are dissolved in alkane solvent at the start of the subsequent repeated second crystallization step.

8. The method of claim 1, further comprising:
repeating the third crystallization step one or more times, wherein the crystals formed at the conclusion of each third crystallization step are dissolved in alkane solvent at the start of the subsequent repeated third crystallization step.

9. The method of claim 1, further comprising:
repeating the third crystallization step until the third crystals have a cannabidivarin content of greater than 97 wt. %, wherein the crystals formed at the conclusion of each third crystallization step are dissolved in alkane solvent at the start of the subsequent repeated third crystallization step.

10. A method of preparing a cannabidivarin isolate, comprising:
performing one or more purification processes on a distillate comprising cannabidivarin until the distillate has a cannabidivarin content greater than a threshold content; and
performing a crystallization step, the crystallization step, comprising:
dissolving the purified distillate in alkane solvent to thereby form a solution; and
cooling the solution to within a temperature range of from about −40° C. to about 60° C. to thereby form crystals comprising cannabidivarin.

11. The method of claim 10, further comprising:
repeating the crystallization step, wherein the crystals formed at the conclusion of each crystallization step are dissolved in alkane solvent at the start of the subsequent repeated crystallization step.

12. The method of claim 11, wherein the crystallization step is repeated until the cannabidivarin content of the crystals is greater than 97 wt %.

13. The method of claim 10, wherein the distillate is a full spectrum distillate obtained from hemp extract.

14. The method of claim 10, wherein the temperature range is from about −20° C. to about 20° C.

15. The method of claim 9, wherein performing one or more purification processes comprises performing chromatography on the distillate.
